(19) United States
(12) Patent Application Publication
(10) Pub. No.: US 2003/0084900 A1
(43) Pub. Date: May 8, 2003

(54) Respiratory assistance apparatus with two-stage turbine

(76) Inventors: Daniel Leclerc, Paris (FR); Laurent Preveyraud, Velizy (FR); Nourreddine Kissi, Meudon La Foret (FR); Delphine Brunner, Antony (FR); Francois Aubatier, Saint Remy Les Chevreuse (FR)

Correspondence Address: Linda K. Russell, Air Liquide, Suite 1800, 2700 Post Oak Blvd., Houston, TX, 77506, US

(21) Appl. No.: 10/269,026

(22) Filed: Oct. 10, 2002

(30) Foreign Application Priority Data
Oct. 10, 2001 (FR) 0113042

(51) Int. Cl. A61M 16/00

(52) U.S. Cl. 128/204.18

(57) ABSTRACT

The invention relates to a respiratory assistance apparatus equipped with a turbine or compressor making it possible to deliver pressurized gas comprising a casing (1) having a gas inlet orifice (2), a motor (6) having a motor shaft (5) of axis (AA), the said motor shaft (5) being able to move about the axis (AA) when it is driven by the said motor (6), a first propeller (3) and a second propeller (4) mounted securely to the said motor shaft (5), an internal partition (7), arranged in the said casing (1), inserted between the two propellers (3, 4) so as to form a first stage (13) internal to the said casing (1) containing the first propeller (3) and a second stage (14) internal to the said casing (1) containing the second propeller (4), the said first stage (13) and second stage (14) being arranged in series, the said internal partition (7) comprising an opening (8) traversed by part of the motor shaft (5) and through which the first internal stage (13) is in fluid communication with the second internal stage (14), and a gas outlet orifice (15) for delivering pressurized gas.

The invention relates to a patient's respiratory assistance or artificial ventilation apparatus equipped with a two-stage turbine (or compressor) making it possible to generate a gas with a non-zero pressure and flow rate, which gas is subsequently distributed to the upper airways of an individual.

In respiratory assistance or artificial ventilation apparatuses, it is normal to use one or more turbines or compressors in order to generate the gas, at the desired pressure and flow rate, intended to be administered to the patients, the terms “turbine” and “compressor” being completely equivalent.

The maximum pressures reached by these types of apparatuses are normally about 50 to 60 hPa.

In some known apparatuses, two medium-pressure turbines are mounted in series so that they can reach the desired gas pressure. In this case, the outlet of the first turbine is connected to the inlet of the second turbine such that the two pressures are added together to form a resulting pressure corresponding to the total desired pressure.

The main advantage of this arrangement is that it makes it possible to regulate pressure by simply varying the speed of the turbines by virtue of their low inertia.

However, conversely, the resulting drawbacks are that this solution requires complete synchronization of the speed of the two turbines, heavy and expensive acoustic insulation, considerable and detrimental heating of the gas due to its passage through the two turbines, and operation under pure oxygen is not possible since the turbines do not allow it.

Moreover, there are also systems similar to low-pressure compressors. Such a system comprises two stages connected in parallel in order to reach the desired flow rate. The entire unit is usually made of a machined aluminium alloy, which makes it possible for it to operate with pure oxygen.

However, although such a system leads to a small overall size, low noise, reduced consumption and allows operation with a gas made up of 100% O₂, it appears that it also has the drawbacks of being expensive since everything is machined then mounted, and of having considerable inertia of the rotating metal parts, which considerably increases the response time of the apparatus.

The aim of the invention is therefore to provide a patient's assisted ventilation apparatus fitted with a turbine which is improved with respect to the known apparatuses with turbines or compressors, that is to say a ventilator with a turbine which is able to generate, from a low-voltage electrical supply, conventionally of about 6 volts to 24 volts, a gas pressure from 0 to 100 hPa; which is able to operate with a minimum flow rate of 120 l/min at 100 hPa; to use ambient air or air enriched with O₂ (up to 100%), and having an overall size and noise which are as low as possible.

The solution of the invention is therefore a respiratory assistance or artificial ventilation apparatus comprising a turbine, in particular a respiratory assistance apparatus comprising a mixing compartment making it possible to mix a first gas with oxygen and a turbine arranged downstream of the said gas mixing compartment, the said turbine making it possible to deliver pressurized gas, comprising:

a casing having a gas inlet orifice,

a motor having a motor shaft of axis (AA), the said motor shaft being able to move about the axis (AA) when it is driven by the said motor,

a first propeller and a second propeller mounted securely to the said motor shaft,

an internal partition, arranged in the said casing, inserted between the two propellers so as to form a first stage internal to the said casing containing the first propeller and a second stage internal to the said casing containing the second propeller, the said first stage and second stage being arranged in series, the said internal partition comprising an opening traversed by part of the motor shaft and through which the first internal stage is in fluid communication with the second internal stage, and

a gas outlet orifice for delivering pressurized gas.

Depending on the case, the apparatus of the invention may comprise one or more of the following technical features:

the gas inlet orifice of the turbine communicates with the first internal stage of the casing, preferably the inlet orifice is of axis (AA);

the gas outlet orifice of the turbine for delivering pressurized gas communicates with the second internal stage of the casing;

the motor of the turbine is an electric motor, preferably a motor powered with low-voltage current;

the internal partition of the turbine is secured to the internal wall of the casing, preferably the internal partition and the casing are made of injection-moulded plastic;

the two propellers of the turbine are identical or different, preferably, the two propellers are fitted with blades and without a flange,

the motor is arranged outside the casing;

the wall of the second stage of the casing of the turbine, located facing the motor, comprises a passage traversed by the motor shaft of the said motor, preferably sealing means arranged around the said passage make it possible to prevent or to minimize any outlet of gas through the said passage;

it comprises a first gas line capable of conveying air, the said first gas, into the mixing compartment so as to supply the compartment with air, the said first gas;

it comprises a second gas line capable of conveying oxygen into the mixing compartment so as to supply the said mixing compartment with oxygen, the second gas line comprising:

a valve with proportional opening making it possible to control the flow rate of oxygen flowing in the said second gas line; and

an oxygen flow rate sensor making it possible to measure the flow rate of oxygen flowing in the said second gas line; and

a flow rate sensor measuring the total flow rate of the gas mixture;

the valve with proportional opening is a solenoid valve;

the valve with proportional opening is controlled by control means connected to the said valve and/or the oxygen flow rate sensor is connected to the control means.

The invention will now be described in more detail by means of the description given below with reference to the appended figures.

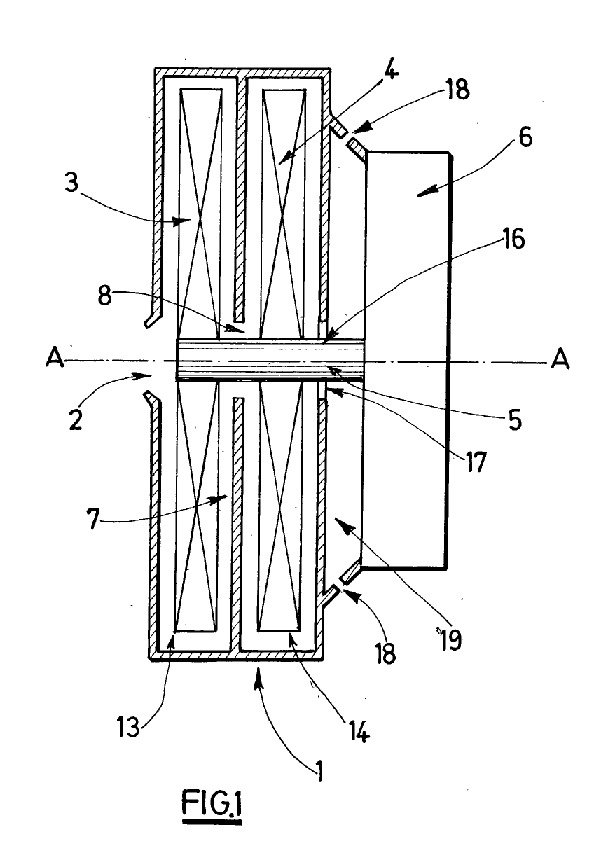

FIG. 1 shows an outline diagram of the architecture (in section) of a turbine equipping an apparatus according to the invention which is a compact motor generator with a flow rate and pressure designed to be used in order to ventilate a patient with ambient air or air enriched with oxygen (up to 100%) and powered by a low-voltage current, typically 24 volts. Within the scope of the invention, the terms gas turbine or compressor are used interchangeably and in a completely equivalent manner.

The turbine is able to deliver a pressure of 0 to 100 hPa with a flow rate of 120 l/min at maximum pressure. The propellers 3, 4 and the casing 1 are produced from an injection-moulded filled plastic, having a high ignition point compatible with the oxygen.

This turbine is connected to a soundproofed enclosure or casing 1 which acts as an inlet, the motor 6 being on the outside. A supply of oxygen in an amount suitable for the inside of this volume makes it possible, upstream of the inlet 2 of the turbine, to make an air/O₂ mixture at the reference value which is insufflated to the patient via the turbine.

The turbine is a system with two stages 13, 14 in series operating by recycling the gas from the first stage 13 into the second 14 by means of a suitable device. By virtue of this technology, the maximum performance is reached at a low speed of about 15 000 rpm. This makes it possible to reduce the noise and extend the life.

A sealing system 17 on the motor shaft 5 prevents any outlet of gas from the propeller/casing assembly (13, 14, 1) towards the outside or into the motor 6.

A decompression chamber 19 between the motor 6 and the turbine makes it possible, via suitable discharge orifices 18, to discharge oxygen should the sealing system 17 fail.

The drive motor 6 is of the brushless, three-phase, 4-pole type having an external rotor with three Hall-effect sensors giving the rotation signal. It is mounted at the end of the motor shaft 5.

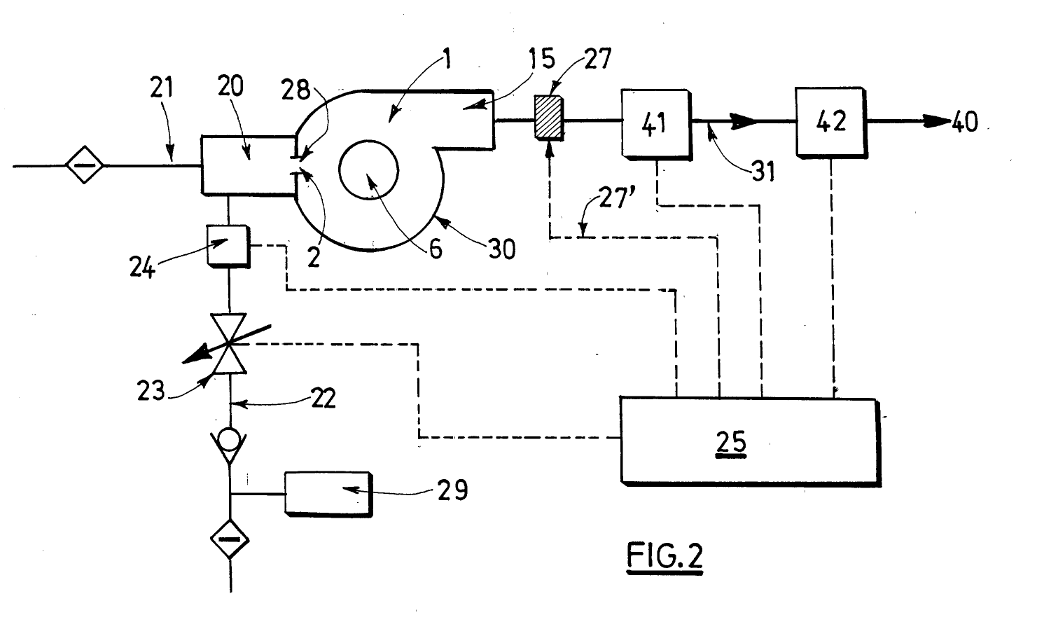

FIG. 2 shows a respiratory assistance apparatus 30 equipped according to the invention with such a turbine, upstream of which a mixing compartment 20 or mixer is arranged for mixing and homogenization in order to mix air with the oxygen coming, for example, from an oxygen duct 21 of a hospital network or from a bottle filled with oxygen.

As for the air in the mixture, this may either also come from a duct 22 of purified air from a hospital network, or be taken directly from the atmosphere; however, in the latter case, it is recommended for it to be submitted to prior treatment in order to purify it, for example filtration of dust and of microorganisms which may be found therein.

The air is conveyed by a first gas line 21 which supplies the mixing compartment 20, while a second gas line 22 makes it possible to convey oxygen so as to supply the mixing compartment 20 with this gas so as to mix the two gases in the desired proportions.

In this case, the second gas line 22 has a valve 23 with proportional opening, preferably a solenoid valve, which can be supplied directly by the hospital network or by a reservoir, at pressures located between about 2.8 and 6 bar, making it possible to control the flow rate of oxygen flowing in the second gas line 22, a pressure sensor 29 and an oxygen flow rate sensor 24 making it possible to measure the flow rate of oxygen flowing in this second line 22.

The valve 23 with proportional opening is controlled by control means 25 connected to the said valve 23.

Similarly, the oxygen flow rate sensor 24 is connected to the drive means 25.

To provide a reliable determination of the oxygen flow rate, the oxygen flow rate sensor 24 is connected to the second gas line 22 between the valve 23 and the mixing compartment 20.

Furthermore, a third gas line 28, not detailed, makes it possible to extract, from the mixing compartment 20, at least part of the gaseous air and oxygen mixture in the desired proportions produced therein and then to supply the turbine 30 according to the invention, via its inlet orifice 2.

The entire system of the invention is controlled by control means 25 comprising a computer or a microprocessor.

To obtain the desired gas mixture, for example containing from 20% to about 99% by volume of oxygen, the flow rate of oxygen supplying the mixing compartment 20 is controlled by means of the proportional valve 23 and the flow rate sensor 24.

Moreover, the gas line 31 also comprises a sensor 27 for sensing the flow rate of air or of a mixture, also connected to the control means 25.

The gaseous mixture enriched with oxygen in the mixing compartment 20, then compressed by the turbine 30 of the invention is then sent to the patient 40 via a patient pipe 31 connected to the outlet of the turbine 30, which patient pipe 31 comprises a mixture flow rate sensor 27, an inspirational valve 41 and an inspirational flow rate sensor 42, all three connected to the control means 25.

The use of injection-moulded plastic parts and of a three-phase motor lead to a low-cost turbine, with a saving in weight and volume making it possible to replace the much more noisy turbines.

1. Respiratory assistance or artificial ventilation apparatus comprising a turbine making it possible to deliver pressurized gas, comprising:
a casing (1) having a gas inlet orifice (2),
a motor (6) having a motor shaft (5) of axis (AA), the said motor shaft (5) being able to move about the axis (AA) when it is driven by the said motor (6),
a first propeller (3) and a second propeller (4) mounted securely to the said motor shaft (5),
an internal partition (7), arranged in the said casing (1), inserted between the two propellers (3, 4) so as to form a first stage (13) internal to the said casing (1) containing the first propeller (3) and a second stage (14) internal to the said casing (1) containing the second propeller (4), the said first stage (13) and second stage (14) being arranged in series, the said internal partition (7) comprising an opening (8) traversed by part of the motor shaft (5) and through which the first internal stage (13) is in fluid communication with the second internal stage (14), and
a gas outlet orifice (15) for delivering pressurized gas.

2. Apparatus according to claim 1, characterized in that the gas inlet orifice (2) of the turbine communicates with the first internal stage (13) of the casing (1), preferably the inlet orifice (2) is of axis (AA):

3. Apparatus according to either of claims 1 and 2, characterized in that the gas outlet orifice (15) of the turbine for delivering pressurized gas communicates with the second internal stage (14) of the casing (1).

4. Apparatus according to one of claims 1 to 3, characterized in that the motor (6) of the turbine is an electric motor, preferably a motor (6) powered with low-voltage current.

5. Apparatus according to one of claims 1 to 4, characterized in that the internal partition (7) of the turbine is secured to the internal wall of the casing (1), preferably the internal partition (7) and the casing (1) are made of injection-moulded plastic.

6. Apparatus according to one of claims 1 to 5, characterized in that the two propellers (3, 4) of the turbine are identical or different.

7. Apparatus according to one of claims 1 to 6, characterized in that the motor (6) of the turbine is arranged outside the casing (1).

8. Apparatus according to one of claims 1 to 7, characterized in that the wall of the second stage (14) of the casing (1) of the turbine, located facing the motor (6), comprises a passage (16) traversed by the motor shaft (5) of the said motor (6), preferably sealing means (17) arranged around the said passage (16) make it possible to prevent or to minimize any outlet of gas through the said passage (16).

9. Apparatus according to one of claims 1 to 8, characterized in that the two propellers (3, 4) of the turbine are fitted with blades and without a flange.

10. Respiratory assistance apparatus according to one of claims 1 to 9, comprising a mixing compartment (20) making it possible to mix a first gas with oxygen, the said turbine (30) being arranged downstream of the said gas mixing compartment (20).

11. Apparatus according to claim 10, characterized in that it further comprises:
a first gas line (21) capable of conveying air, the said first gas, into the mixing compartment (20) so as to supply the compartment (20) with air, the said first gas,
a second gas line (22) capable of conveying oxygen into the mixing compartment (20) so as to supply the said mixing compartment (20) with oxygen, the second gas line (22) comprising:
a valve (23) with proportional opening making it possible to control the flow rate of oxygen flowing in the said second gas line (22), and
an oxygen flow rate sensor (24) making it possible to measure the flow rate of oxygen flowing in the said second gas line (22), and
a flow rate sensor (27) measuring the total flow rate of the gas mixture.

12. Apparatus according to claim 11, characterized in that the valve (23) with proportional opening is a solenoid valve and/or the valve (23) with proportional opening is controlled by control means (25) connected (23′) to the said valve (23) and/or the oxygen flow rate sensor (24) is connected (24′) to the control means (25).
